(19) United States
(12) Patent Application Publication
(10) Pub. No.: US 2007/0197984 A1
(43) Pub. Date: Aug. 23, 2007

(54) Fecal disposal device

(76) Inventors: William C. Richardson, St. Louis, MO (US); Cynthia Denise Richardson, St. Louis, MO (US); Clorinda J. Blanchard, Conyers, GA (US)

Correspondence Address: POLSTER, LIEDER, WOODRUFF & LUCCHESI, 12412 POWERSCOURT DRIVE SUITE 200, ST. LOUIS, MO, 63131-3615, US

(21) Appl. No.: 11/357,569

(22) Filed: Feb. 17, 2006

(51) Int. Cl. A61F 5/44

(52) U.S. Cl. 604/348; 604/385.19

(57) ABSTRACT

The present invention relates to a disposable fecal retaining device which has a substantially rectangular pad having an exterior moisture impermeable layer juxtaposed to an absorbing layer. The device is configured to provide a pouch with the absorbing layer on the inside of the pouch. The device has an entry port substantially surrounded by tape for adhesion of the device about the anal opening of a wearer to hold the device in place.

### CROSS-REFERENCE TO RELATED APPLICATIONS

Not Applicable

### STATEMENT REGARDING FEDERALLY SPONSORED RESEARCH

Not Applicable

### BACKGROUND OF THE INVENTION

When a person is disabled, whether it be permanently or temporarily, and cannot use a toilet or a bed pan, conventional disposable incontinent products may be used by the person. The disposable products are usually the standard adult diaper which consists of a liquid impermeable exterior, such as a film, an absorbent layer and a liquid permeable facing material. Liquid permeates the facing and hopefully is trapped by the absorbent layer. Although these disposable products generally hold discharges of urine, they are not satisfactory as designed for holding fecal matter.

Attempts have been made to provide disposable products which can be used for incontinence and also claim to hold fecal matter. An example is U.S. Pat. No. 6,423,884 B1 to Heidi Ann Ochmen. This patent provides an absorbent article including an outer cover and a bodyside liner. The article has one or more defined apertures sized to retain exudates from a typical bowel movement in an attempt to displace such exudates from the skin of the wearer. The structure has a surface layer on the outer surface and a bodyside liner which contacts the body of the user. The bodyside liner has a slick surface to assist in moving fecal material into the defined apertures. However, while moving the fecal material into the defined apertures, the fecal material is in contact with the skin of the wearer. Even though the article also contains an absorbent core, mounted particularly in the front portion of the article, the fecal material is not quickly contained so as not to irritate the skin of the wearer.

Many disabled persons sleep on their stomachs or their sides so an article such as that just described, does not keep the fecal matter away from the skin of the wearer.

The disposable fecal containment device of the present invention overcomes these problems by the deposition of the fecal matter away from the skin of the wearer immediately as exudation of the fecal matter takes place.

### BRIEF SUMMARY OF THE INVENTION

The present invention provides a disposable fecal matter retaining device which has a substantially rectangular shaped pad with an exterior liquid impermeable layer juxtaposed to an absorbing layer. The pad is configured to provide a pouch with the absorbing layer on the inside of the pouch. The pad has an entry port (aperture) substantially surrounded by adhesive tape placed on the liquid impermeable side of the pad to allow for adhesion of the device to the area surrounding the anal opening of the wearer to hold the device in place. As the fecal matter exudates through the anal opening, it is quickly received and retained by the pouch, hence the fecal matter is not in contact with the skin of the wearer.

The absorbent pad generally is comprised of three juxtaposed layers.

The exterior layer is a liquid or moisture impermeable film. Juxtaposed adjacent to the film is an absorbent layer such as that found in disposable adult and baby diapers. This absorbent layer frequently is made from wood pulp fibers air laid on a flat surface. The absorbent layer may or may not have super absorbent material incorporated therein. The absorbent layer is of smaller dimensions than the liquid impermeable layer so that a border of the liquid impermeable layer surrounds the absorbent layer.

Juxtaposed to the absorbent layer on the side opposite the liquid impermeable layer is a liquid permeable facing layer which most generally is a nonwoven fabric or the like. The facing layer extends beyond the dimensions of the absorbent layer and has substantially the same dimensions as those of the liquid impermeable layer and is adhered to the liquid impermeable layer about the perimeter of the absorbent pad. The absorbent layer may be adhered to the liquid impermeable layer if so desired.

The absorbent pad is configured to provide a pouch having the facing layer and/or the absorbent layer on the interior of the pouch. The entry port is of sufficient size to accommodate the fecal material as it exudes from the rectum and enters the pouch. The entry port (aperture) may be oval, rectangular, circular, V-shaped or any satisfactory shape.

### BRIEF DESCRIPTION OF THE SEVERAL VIEWS OF THE DRAWINGS

In the accompanying drawings which form part of the specification:

FIG. 1 is a perspective view of one embodiment of the present invention;

FIG. 2 is a front elevational view of the embodiment of FIG. 1;

FIG. 3 is a rear elevational view of the embodiment of FIG. 1;

FIG. 4 is a side elevational view of the embodiment of FIG. 1;

FIG. 5 is a perspective view of the embodiment of FIG. 1 when worn by a person lying down;

FIG. 6 is a perspective view of another embodiment of the present invention;

FIG. 7 is a front elevational view of the embodiment of FIG. 6;

FIG. 8 is a rear elevational view of the embodiment of FIG. 6;

FIG. 9 is a cross-sectional view along lines 9-9 of FIG. 8; and

FIG. 10 is a perspective view of the embodiment of FIG. 6 when worn by a person lying down.

Corresponding reference numerals indicate corresponding parts throughout the several figures of the drawings.

### DETAILED DESCRIPTION OF THE INVENTION

The following detailed description illustrates the invention by way of example and not by way of limitation. The description clearly enables one skilled in the art to make and use the invention, describes several embodiments, adaptations, variations, alternatives, and uses of the invention, including what is presently believed to be the best mode of carrying out the invention.

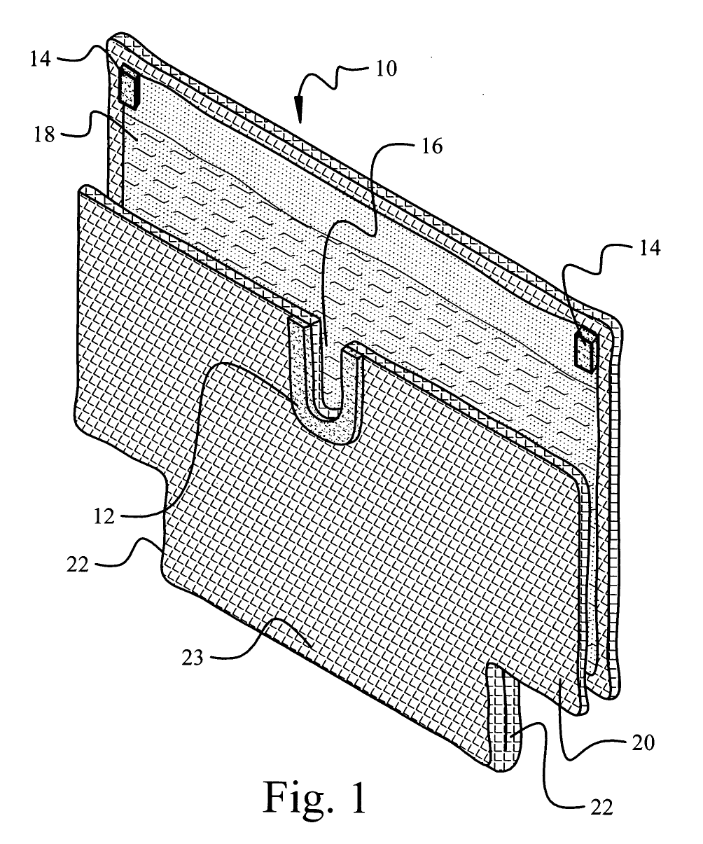

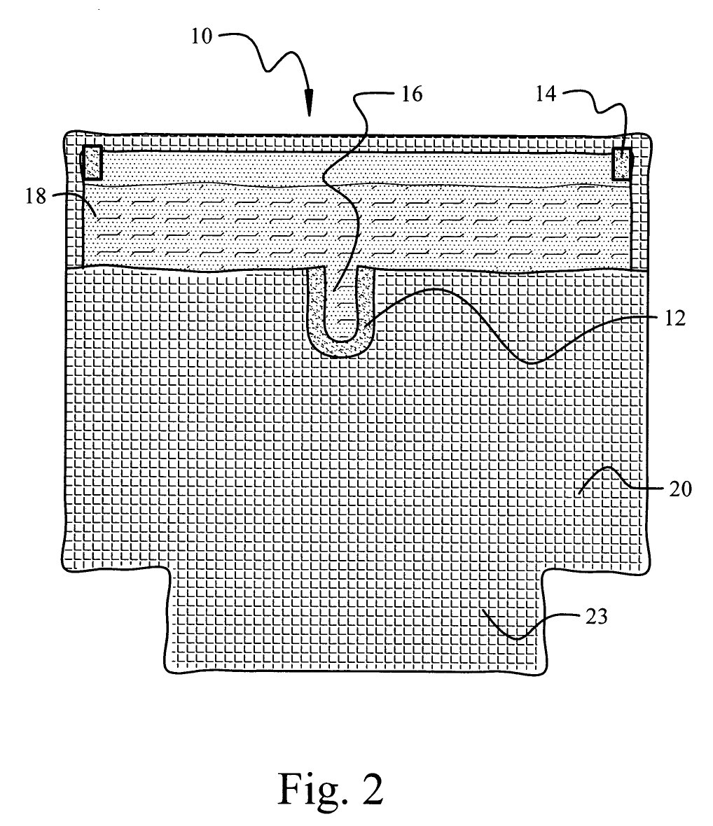

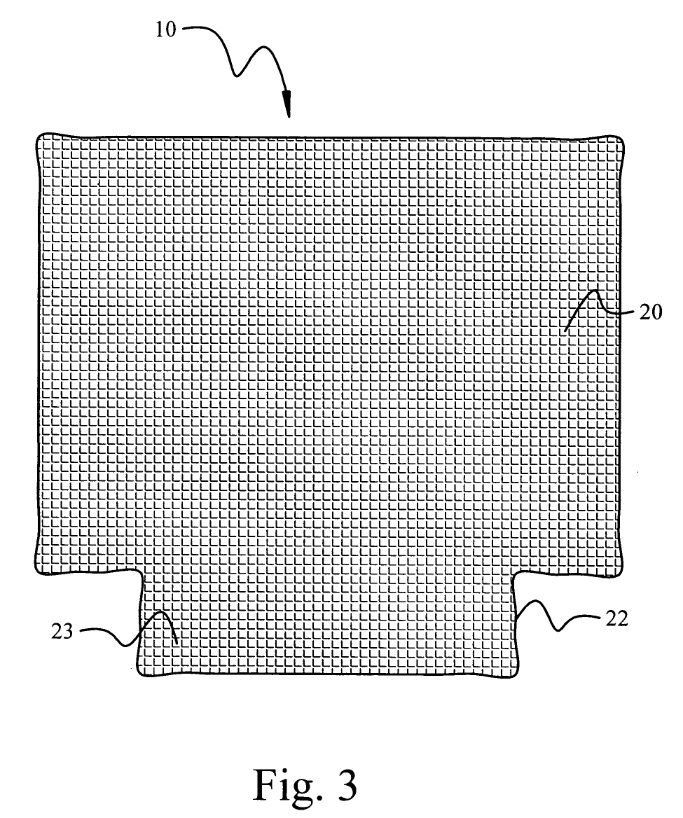

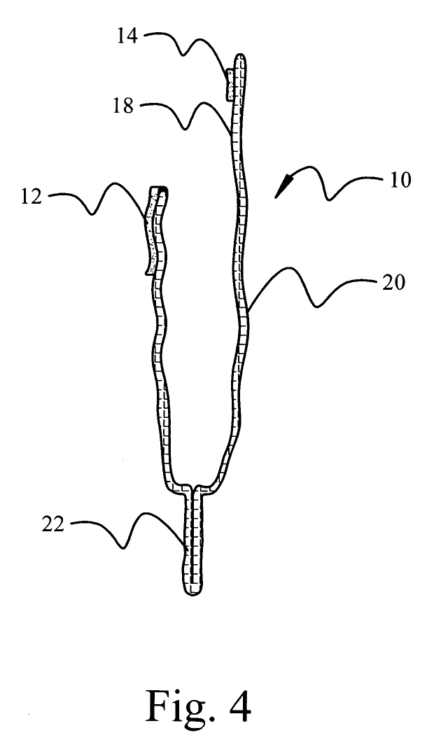

FIG. 1 is a perspective view of one embodiment of the absorbent pad device 10 of the present invention. FIGS. 2-4 provide different views of the pad 10. An absorbent pad 10, as described above, has adhesive tape 12 around an aperture 16. The adhesive tape 12 is covered with a removable plastic strip (not shown) so as to expose the adhesive when applying the aperture 12 of the pad 10 to the skin of the wearer about the anal opening. The aperture 12 may be V-shaped or U-shaped or any other convenient configuration.

A similar tape 14 is applied to the upper corners of the pad 10 for fastening the corners of the pad 10 to the person's torso in order to maintain the pad 10 in the proper position when worn.

The pad 10 has an exterior 20 which is liquid impermeable and preferably is a film type material. The interior 18 of the pad 10 is a liquid permeable material, e.g., a non-woven material, which is soft and pleasing to the skin of the wearer. Between the exterior 20 of the pad 10 and the interior facing 18 is an absorbent substance (not shown) such as an absorbent batt of wood pulp fibers, or superabsorbent, or a mixture thereof, or the like.

When applied to the wearer, a portion of the facing 18 comes into contact with the skin of the wearer whereas the liquid impermeable film 20 remains on the exterior of the pad 10.

The lower portion of the pad 10 forms a pouch 23 which holds the fecal excrement. The pouch 23 is sealed along sides 22 to prevent leakage.

Whether the person wearing the pad 10 is lying on his/her stomach or in a left or right lateral decubitis position, the pad 10 contains the fecal matter with little or no contact with the skin. Thus, if a patient has a catheter and needs to have a bowel movement, it no longer is necessary for the patient to have to situate himself/herself on a bedpan, but instead, the pad 10 of the present invention is easily applied when the patient is lying on the stomach or in a left or right decubitis position.

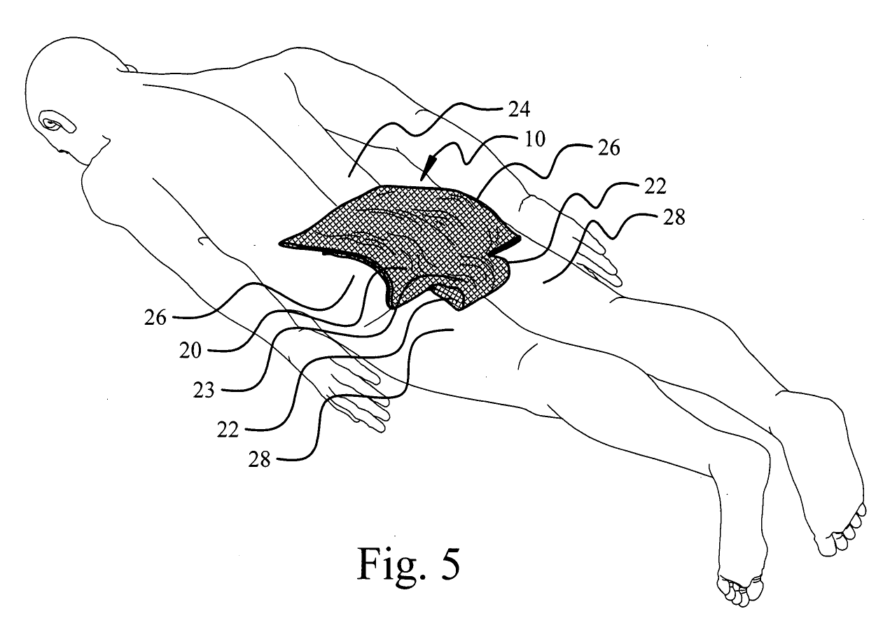

Referring to FIG. 5, there is an example of the pad 10 applied to a human torso 24. The aperture 16 is not shown, but the tape 14 about the aperture 16 is applied around the anal opening (also not shown) between the buttocks 26. The pouch 23 is situated away from the buttocks 26 toward the legs 28. The liquid impermeable film covers the exterior 20 of the pad 10. It is easier to apply the pad 10 when the person is lying on his/her stomach, however, the pad 10 may be applied when the person is in the left or right decubitis position, for example, following abdominal surgery.

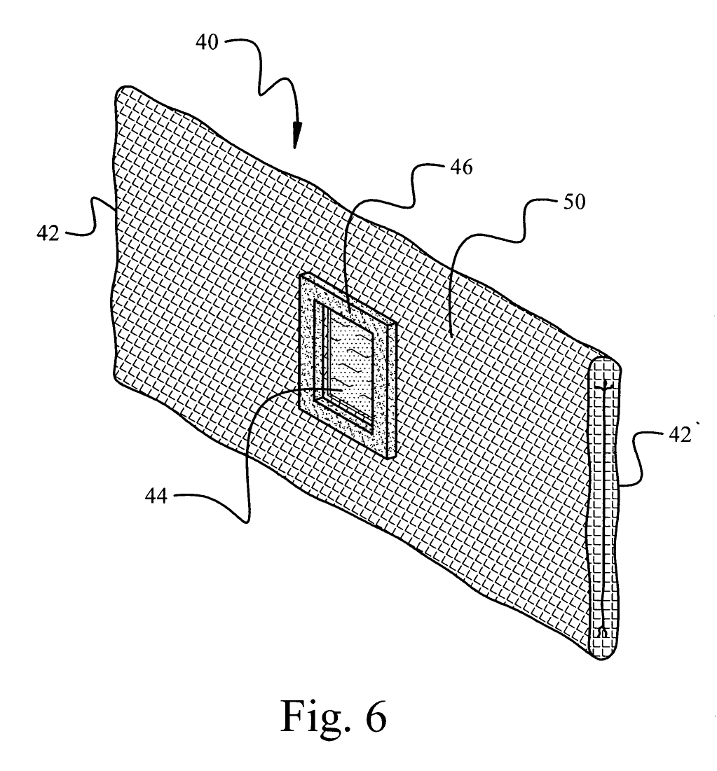

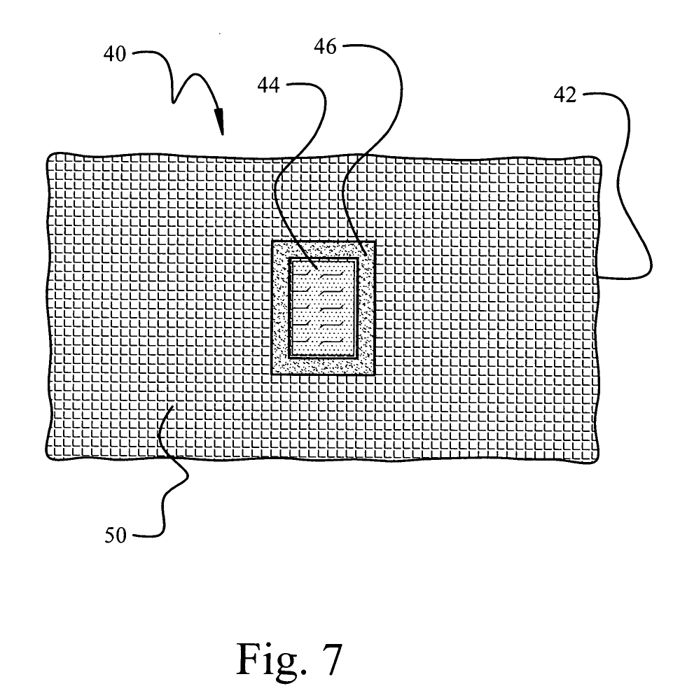

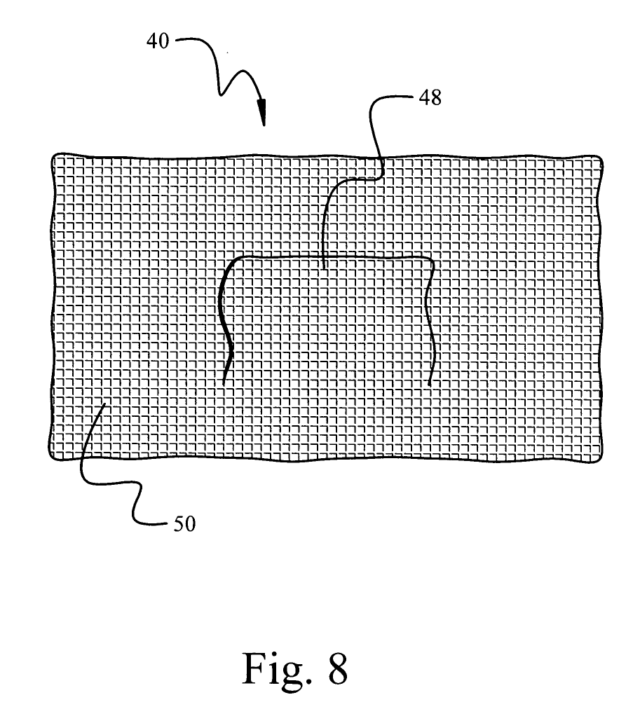

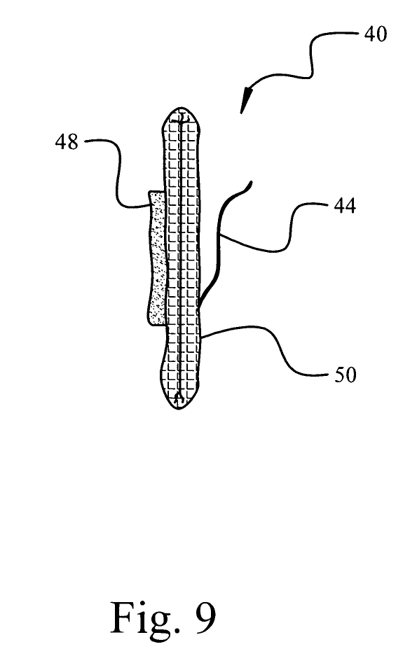

FIG. 6 is a perspective view of another embodiment of an absorbent pad device 40 of the present invention. FIGS. 7-9 provide different views of the pad device 40. An absorbent pad 40 has adhesive tape 46 around an aperture 44. The adhesive tape 46 is covered with a removable plastic strip (not shown) so as to expose the adhesive when applying the aperture 44 of the pad 40 about the anal opening of the wearer.

The aperture 44 may be shaped as a square or rectangle, or an oval shape or any other convenient configuration.

The pad 40 is configured from a typical disposable commercial pad having a liquid impermeable film for the exterior, an absorbent layer juxtaposed to the film and a liquid permeable facing layer juxtaposed to the absorbent layer on the side opposite the liquid impermeable film. Usually these commercial pads are rectangular in shape.

An end of a rectangular shaped pad 40 is folded over the facing layer generally less than about half of the length of the pad 40. The other end of the pad 40, which becomes the lower portion of the pad 40, is folded over the facing layer so as to somewhat overlap the previously folded-over portion. The overlapped portions 45 and 47 are sealed. Two parallel cuts are made at the end of the sealed portions 45 and 47, to form a trap door 48 to allow inspection of the interior of the pouch formed below the trap door 48 when the pad 40 is fastened to the wearer.

The pad 40 is sealed about the edges 42 with the liquid absorbent side facing inward and the liquid impermeable film on the exterior 50. On one side of the exterior 50 an aperture 44 is provided with adhesive 46 surrounding the aperture 44. The adhesive tape 46 is covered with a removable plastic strip (not shown) so as to expose the adhesive when applying the aperture 44 of the pad 40 about the anal opening of the wearer.

On the side opposite the aperture 44 of the exterior 50 of the pad 40, is a “trap door” 48 which may be opened to assist in viewing the aperture 44 as the pad 40 is being adhesively attached with the aperture 44 about the anal opening of the wearer. Furthermore, the trap door 48 may be opened for inspection of the interior of the pad 40 to see if a bowel movement has occurred.

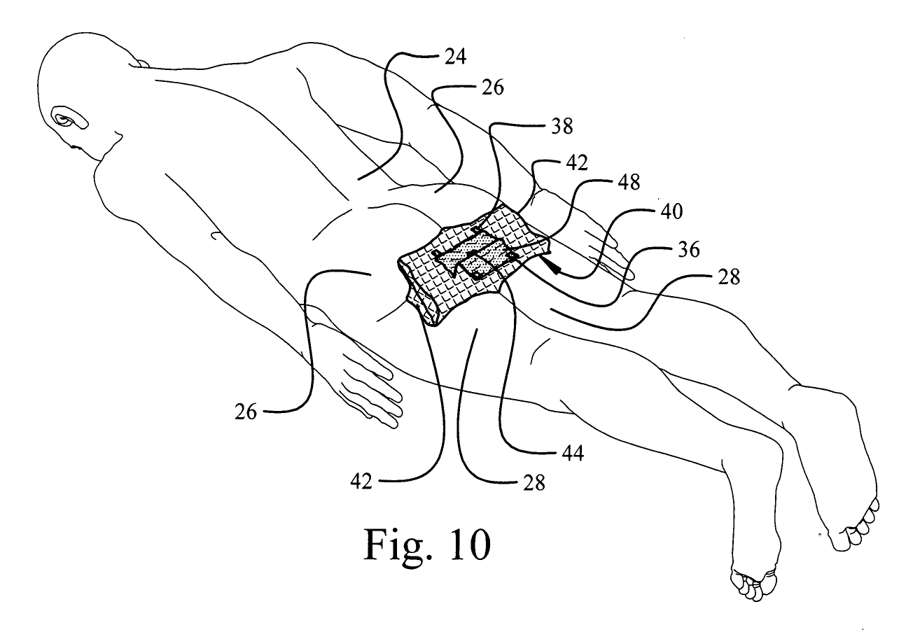

Referring to FIG. 10, there is an example of the pad 40 applied to a human torso 24. The aperture 44 is not shown, but the adhesive tape 46 about the aperture 44 is applied around the anal opening (also not shown) between the buttocks 26. The liquid impermeable film covers the exterior 50 of the pad 40. It is easier to apply the pad 40 when the person is lying on his/her stomach, however, the pad 40 may be applied when the person is in the left or right decubitis position, for example, following abdominal surgery. The trap door 48 is depicted in an open position revealing a portion of the aperture 44. Adhesive tabs 36 having releasable and reusable adhesive attach to landing sites 38 so as to again close the trap door 48.

The pad 40 tends to form a pouch or envelope when adhesively attached to a person about the anal opening. The added feature of a trap door 48 is optional for the pad 40.

Each of the pads 10 and 40 provide many advantages over known commercial pads. Each of the pads 10 and 40 is easily applied to a patient who is lying down on his/her stomach or in a left or right lateral decubitis position.

The absorbent pad device 10 or 40 of the present invention protects the skin of the wearer because the fecal matter as it exudates from the anal opening is immediately deposited in the pouch of the device so that contact with the skin of the wearer is at a minimum. This helps to prevent diaper rash or other skin irritation.

Another advantage of the present invention is that the device contains the feces in the pouch of the device so that disposal of the feces is easily accomplished. When wearing the device of the invention, accidents where the feces get into the bed clothes are almost eliminated.

The absorbent portion of the device absorbs and contains any liquid emitted from the feces thereby keeping the patient dry. The ready containment and absorption by the device of the present invention provides better sanitary conditions for both the caretaker and the patient by making clean up simple and quick. Also the wearer may keep the device on for long periods of time without chafing or irritation of the skin.

In view of the above, it will be seen that the several objects of the invention are achieved and other advantageous results are obtained. As various changes could be made in the above constructions without departing from the scope of the invention, it is intended that all matter contained in the above description or shown in the accompanying drawings shall be interpreted as illustrative and not in a limiting sense.

1. A disposable fecal retaining device with a pouch comprising a substantially rectangular shaped pad having an exterior moisture impermeable layer juxtaposed to an absorbing layer, the pad being configured to provide a pouch with the absorbing layer on the inside of the pouch, the pad having an entry port substantially surrounded by tape for adhesion of the device about the anal opening of a wearer to hold the device in place.

2. The device of claim 1 wherein the pad has a moisture permeable facing layer juxtaposed to the absorbing layer on the side opposite the impermeable layer.

3. The device of claim 2 wherein the moisture impermeable layer and the facing layer extend beyond the edges of the absorbing layer, the moisture impermeable layer and the facing layer being adhesively bonded about the edges of the absorbent layer.

4. The device of claim 1 wherein the pouch is configured by folding one end of the pad a portion of the way toward the other end of the pad, keeping the absorbent layer facing inward and sealing the sides of the folded over portion of the pad thereby forming a flap with the unfolded end of the pad.

5. The device of claim 4 wherein the flap is held in place on the wearer by adhesive tabs.

6. The device of claim 1 wherein the pouch is configured by folding one end of the rectangular shaped pad over the absorbent layer less than about one half the length of the pad, folding a second end of the pad over the facing layer overlapping the previously folded-over portion sufficiently to allow sealing of the overlapped portions, and sealing the side edges of the pad to form a pouch.

7. The device of claim 6 wherein a trap door is formed by two parallel cuts made substantially at the center of the end of the second folded over portion before sealing the folded over portion to form a trap door which trap door has adhesive tabs for closing the trap door.

8. A disposable fecal retaining device with a pouch comprising a pad having an exterior moisture impermeable layer juxtaposed to an absorbing layer, the pad being configured to provide a pouch with the absorbing layer on the inside of the pouch wherein the pouch is configured by folding the ends of the pad toward each other, partially overlapping the ends to form a flap, keeping the absorbent layer facing inward and sealing the sides of the pad after folding, the pad having an entry port substantially surrounded by tape for adhesion of the device about the anal opening of a wearer to hold the device in place.

9. The device of claim 8 having a trap door to allow inspection of the interior of the pouch of the device.
